(19) United States
(12) Plant Patent Application Publication
(10) Pub. No.: US 2005/0172373 P1
(43) Pub. Date: Aug. 4, 2005

(54) Floribunda rose plant named 'Meiangele'

(50) ROSA HYBRIDA; FLORIBUNDA ROSE

(76) Inventor: Alain A. Meilland, Antibes (FR)

Correspondence Address: BURNS DOANE SWECKER & MATHIS L L P, POST OFFICE BOX 1404, ALEXANDRIA, VA, 22313-1404, US

(21) Appl. No.: 10/767,258

(22) Filed: Jan. 30, 2004

(51) Int. Cl. H02J 7/00; H02J 7/32; H02J 7/35; H01M 10/44; H01M 10/46; A01H 5/00

(52) U.S. Cl. PLT/150

(57) ABSTRACT

A new and distinct variety of rose plant of the Floribunda Class is provided which forms attractive red blossoms commonly having at least one hundred petals that tend to be quartered around the receptacle. The growth habit is bushy. Very dense and leathery dark green glossy foliage is formed that contrasts nicely with the blossom coloration. Very good disease resistance is displayed especially with respect to Black Spot. The new variety is well suited for growing as attractive ornamentation in the landscape.

### BOTANICAL/COMMERCIAL CLASSIFICATION

Rosa hybrida / Floribunda Rose Plant

### VARIETAL DENOMINATION

cv. ‘Meiangele’

### SUMMARY OF THE INVENTION

The new variety of Rosa hybrida Floribunda rose plant was created by artificial pollination wherein two parents were crossed which previously had been studied in the hope that they would contribute the desired characteristics. The female parent (i.e., the seed parent) of the new variety was the ‘Kormax’ variety (non-patented in the United States). The male parent (i.e., the pollen parent) was the product of the cross of the ‘Meideuji’ variety (non-patented in the United States) and the ‘Meidomonac’ variety (U.S. Plant Pat. No. 5,105). The ‘Kormax’ variety is marketed under the ROTE MAX GRAF trademark, and the ‘Meidomonac’ variety is marketed under the BONICA trademark. The parentage of the new variety can be summarized as follows:
‘Kormax’×(‘Meideuji’×‘Meidomonac’).

The seeds resulting from the above pollination were sown and small plants were obtained which were physically and biologically different from each other. Selective study resulted in the identification of a single plant of the new variety.

It was found that the new variety of Floribunda rose plant of the present invention possesses the following combination of characteristics:

(a) exhibits a bushy growth habit,

(b) forms attractive red blossoms commonly having at least 100 petals that tend to be quartered around the receptacle,

(c) forms very dense and leathery dark green glossy foliage, and

(d) exhibits very good resistance to Black Spot.

The new variety well meets the needs of the horticultural industry. It is particularly well suited for growing as attractive ornamentation in the landscape.

The new variety can be readily distinguished from its parental varieties. More specifically, the ‘Kormax’ variety forms dissimilar single blossoms having approximately six petals. The ‘Meideuji’ variety displays a climbing growth habit. The ‘Meidomonac’ variety forms light pink blossoms.

The new variety has been found to undergo asexual propagation by a number of routes including budding, grafting, and the use of cuttings. Asexual propagation by these methods as performed at Le Cannet Des Maures, Var, France, has shown that the characteristics of the new variety are strictly transmissible from one generation to another.

The new variety has been named the ‘Meiangele’ variety.

### BRIEF DESCRIPTION OF THE PHOTOGRAPH

The accompanying photograph shows as nearly true as it is reasonably possible to make the same in a color illustration of this character typical specimens of the plant parts of the new variety. The rose plants of the new variety were approximately one and one-half years of age and were photographed during October while growing outdoors on their own roots at Le Cannet Des Maures, Var, France. Dimensions in centimeters are included at the bottom of the photograph.

FIG. 1 illustrates a specimen of a young shoot;

FIG. 2 illustrates a specimen of a floral bud before the opening of the sepals;

FIG. 3 illustrates a specimen of a floral bud at the opening of the sepals;

FIG. 4 illustrates a specimen of a floral bud at the opening to the petals;

FIG. 5 illustrates a specimen of a flower in the course of opening;

FIG. 6 illustrates a specimen of an open flower—plan view—obverse;

FIG. 7 illustrates a specimen of an open flower—plan view—reverse;

FIG. 8 illustrates a specimen of a fully open flower—plan view—obverse;

FIG. 9 illustrates a specimen of a fully open flower—plan view—reverse;

FIG. 10 illustrates a specimen of a floral receptacle showing arrangement of the stamens and pistils;

FIG. 11 illustrates a specimen of a floral receptacle showing arrangement of the pistils (stamens removed);

FIG. 12 illustrates a specimen of a flowering stem;

FIG. 13 illustrates a specimen of a main branch;

FIG. 14 illustrates a specimen of a leaf with 3 leaflets—plan view—upper surface;

FIG. 15 illustrates a specimen of a leaf with 5 leaflets—plan view—under surface; and

FIG. 16 illustrates a specimen of a leaf with 7 leaflets—plan view—upper surface.

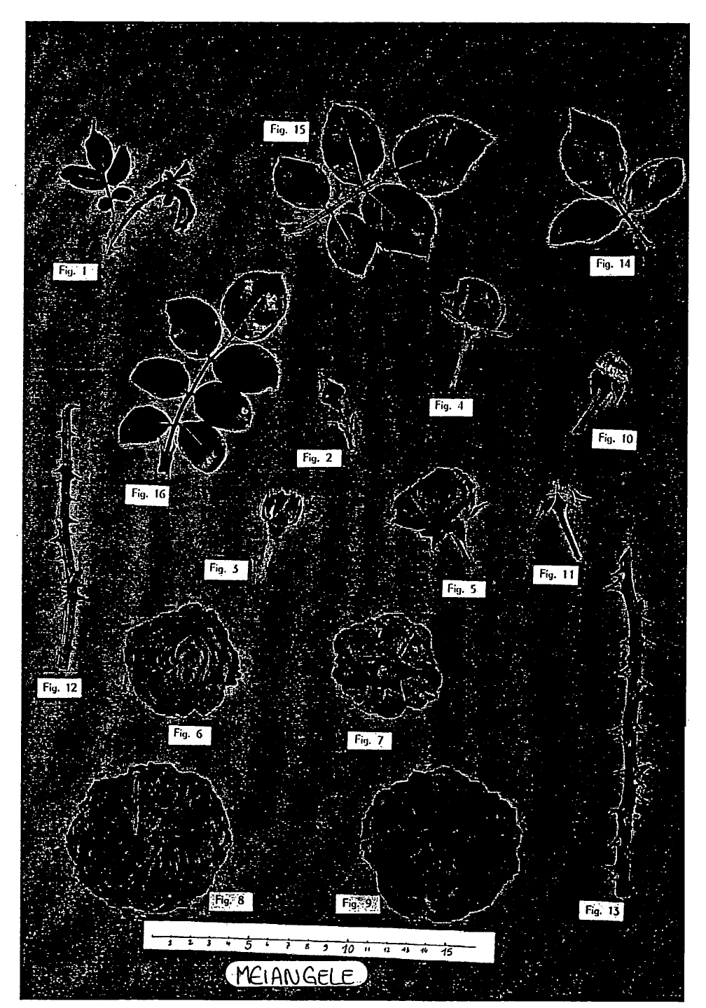

### DETAILED DESCRIPTION

The chart used in the identification of the colors is that of The Royal Horticultural Society (R.H.S. Colour Chart). Common color terms are to be accorded their customary dictionary significance. The description is based on the observation of one and one-half year-old specimens of the new variety which were observed during October while growing outdoors on their own roots at Le Cannet Des Maures, Var, France.
Class: Floribunda.
Plant:
   Height.—approximately 70 cm on average at the end of the growing season.
   Width.—approximately 70 cm on average at the end of the growing season.
   Habit.—bushy.
Branches:
   Thorns.—rather upright and curved downwards on the upper surface and slightly concave on the under surface.
Leaves:
   Arrangement.—alternate and pinnately compound.
   Leaflets.—number: 3, 5 (most often), and 7. — shape: typically elliptical to slightly ovate. — apex: acute to cuspidate. — base: commonly broadly rounded. — texture: leathery. — margin: serrate. — general appearance: very dense, dark green, and glossy.
Inflorescence:
   Number of flowers.—commonly approximately 2 to 15 blooms per stem.
   Peduncle.—rigid and commonly approximately 4 cm in length on average and medium in diameter.
   Sepals.—five in number, and lanceolate commonly with a few small foliaceous extensions.
   Buds.—shape: ovoid. — size: medium. — length: approximately 2 cm on average. — color as calyx breaks: upper petal surface: near Red Group 53C and widely suffused with Red Group 45A. under petal surface: near Red Group 53C. basal petal spot: near Yellow Group 2C.
   Flower.—form: very double. — shape: cup-shaped. — diameter: approximately 9 to 10 cm when fully open. — color when fully open: upper surface: near Red-Purple Group 57C widely suffused with Red-Purple Group 57A. under surface: near Red-Purple Group 57C. basal petal spot: none. — petal number: commonly at least 100, e.g., approximately 100 to 132 on average. — petal arrangement: imbricated and quartered around the receptacle. — petal drop: the petals commonly detach cleanly.
Development:
   Vegetation.—strong.
   Resistance to disease.—very good especially with respect to Black Spot.

1. A new and distinct variety of Floribunda rose plant characterized by the following combination of characteristics:
(a) exhibits a bushy growth habit,
(b) forms attractive red blossoms commonly having at least 100 petals that tend to be quartered around the receptacle,
(c) forms very dense and leathery dark green glossy foliage, and
(d) exhibits very good resistance to Black Spot;
substantially as herein shown and described.
